(19) United States
(12) Patent Application Publication
(10) Pub. No.: US 2023/0240309 A1
(43) Pub. Date: Aug. 3, 2023

(54) NEW MICROBIAL CONTROL OF EDIBLE SUBSTANCES

(71) Applicant: Lactobio A/S, Copenhagen Ø (DK)

(72) Inventors: Charlotte VEDEL, Copenhagen Ø (DK); Ida Blomquist CHRISTENSEN, Copenhagen Ø (DK); Søren KJAERULFF, Copenhagen Ø (DK); Katja Billekop OLSEN, Copenhagen Ø (DK); Sigurd CHRISTENSEN, Copenhagen Ø (DK)

(21) Appl. No.: 18/013,627

(22) PCT Filed: Jul. 1, 2021
(86) PCT No.: PCT/EP2021/068215

§ 371 Date: Dec. 29, 2022

(30) Foreign Application Priority Data
Jul. 2, 2020 (DK) PA 2020 00781
Feb. 11, 2021 (DK) PA 2021 00151
Mar. 19, 2021 (DK) PA 2021 00283
May 21, 2021 (DK) PA 2021 00529

(51) Int. Cl. A23B 4/22; A01N 63/50; A01P 1/00; A23L 3/3463; A23L 3/3508; A23K 30/00; A23K 20/195; A23K 20/105; A23L 13/40

(52) U.S. Cl. CPC A23B 4/22; A01N 63/50; A01P 1/00; A23L 3/34635; A23L 3/3508; A23K 30/00; A23K 20/195; A23K 20/105; A23L 13/428

(57) ABSTRACT

The present invention relates to a composition for control of microbial development of an edible substance the composition comprises a supernatant comprising a bacteriocin produced from fermentation of a bacteriocin producing bacteria and an organic acid produced from fermentation of a bacteriocin producing bacteria, wherein organoleptic characteristics of the edible substance are not significantly altered.

### TECHNICAL FIELD OF THE INVENTION

The present invention relates to the field of improving microbial safety in the production of edible products. In particular the present invention relates to microbial originating composition useful for reducing the amount of pathogenic organisms in edible products. The present invention relates to a new preservative comprising a bacteriocin comprising supernatant. In particular the present invention relates to novel compositions for preservation of edible products.

### BACKGROUND OF THE INVENTION

This invention relates to a composition and a method for preserving an edible substance, and particularly a preservative comprising supernatant from a bacteriocin producing bacteria which do not significantly change the organoleptic characteristics of an edible substance when applied to such edible substance.In the food industry, and particularly in those areas devoted to the processing of foods, preservatives are needed to prevent the growth of spoilage microorganisms and pathogens.

The control of contamination by microorganisms is a recognized problem in the food industry. The preparation of food products, and particularly processed food, fresh meat, processed meat and meat products for the retail market, is largely concerned with the control of microbial contact with food in order to increase the shelf life of food products. Food products having an extended shelf life afford more time in which handlers, shippers, and wholesalers can transport and sell such food before spoilage occurs. Efforts to increase the shelf life of food products, such as processed foods, have traditionally been focused on reducing the number of bacteria present on the surface of the food product by addition of preservatives or use of preservation methods to prolong the retail acceptability of food products. For example, reducing water activity by salts or sugars, vacuum packing of food in gas permeable packages is commonplace. Irradiation with ultraviolet light has been used to reduce the number of microorganisms on food surfaces. Salting of has long been practiced to preserve e.g. meat products. Refrigeration is also widely used to deter the rapid growth of spoilage and pathogenic bacteria in food products. Spoilage bacteria, such as e.g. pseudomonas, lactobacillus and clostridium are known to grow most rapidly at about room temperature. Although such bacteria are present on foods at lower temperatures, their growth is significantly slowed by cooler environments. Mere refrigeration alone, however, is not totally effective in preventing or adequately retarding the growth of spoilage or pathogenic bacteria for any appreciable amount of time.

The shelf life of foods, and in particular meat and processed meat, have also been extended somewhat by the use of chemical agents. Chemical treatment of meat to destroy surface bacteria has traditionally been accomplished by treating meat with weak acids and/or chlorine solutions. These conventional techniques, however, often create undesirable color, flavor and other modifications of meat, and are often ineffective to maintain meat in a saleable condition for any appreciable period of time.

Although the control of spoilage and pathogenic bacterial growth is a recognized problem in the food industry, the reduction of shelf life attendant to such growth continues to be a significant problem. Many techniques have been employed in the past in an effort to destroy surface bacterial flora on foods. For example, U.S. Pat. No. 4,852,216 discloses a disinfection system using an acetic acid spray in order to reduce bacterial levels and thereby increase shelf life of food products. Similarly, U.S. Pat. No. 3,924,044 discloses a method for applying a hot, dilute acid solution to food surfaces to destroy psychotropic spoilage bacteria on food surfaces and others are using non-fermentative bacteria for biopreservation (U.S. Pat. No. 6,569,474B) or antimicrobial peptides (U.S. Pat. No. 8,828,459B). Common for these preservative techniques are that they often are not sufficient to control undesired microorganisms, or they change the organoleptic appearance of the food substance. Either by an un-pleasant odor, taste, dis-colorization or when using live bacteria a slimming appearance caused by the preserving bacteria producing extracellular polysaccharides as well as a change in taste of the edible product.

No prior art techniques have taught the effective elimination of growth of undesired bacteria to achieve a significant extension of shelf life in fresh food products. In e.g. the meat packing industry, many types of bacteria are known to cause food poisoning including: E. coli. Salmonella, Listeria, Staphylococcus, Streptococcus. Bacillus, Campylobacter, Yersinia, Brucella, Chlamydia, Leptospira and Clostridium. These pathogenic bacteria each grow and proliferate under different conditions, any or all of which may be present in a meat processing facility. For example, Listeria is generally found in cool, damp environments such as coolers and processing areas. Staphylococcus is often found on skin and cattle hair, in fecal material, in infected cuts and internal abscesses, and is sometimes associated with poor hygienic practices of food handlers.

Spoilage bacteria, including psychotropic bacteria such as Pseudomonas, Lactobacillus and Coliform, affect the shelf life of meat products by causing discoloration of meat and undesired odors. In the environment of a food processing facility, spoilage bacteria typically proliferate at a greater rate than do pathogenic bacteria or lactic bacteria. It has been recognized that various sanitizing techniques, including acetic acid sprays, application of anti-microbial agents, and irradiation, can be used to reduce the total number of bacteria present in processing plant. Lactic acid bacteria have been used as biopreservatives, however, this group of bacteria typically ferment the food product resulting in different fermented food products which all have significant different taste, look and smell as compared to un-fermented foods. Lactic acid bacteria are also known for generation of gasses during fermentation with a significant contribution to the smell of such fermented product or production of extracellular polysaccharides contributing to an unwanted slimming appearance. It has been a surprise to identify new Lactobacillus strains which are able to have the same antimicrobial and preservative effect as in fermented food, but with-out the fermentation of the edible product just by addition of a supernatant from fermentation comprising metabolites. Wherein such supernatant does not have a negative impact on the organoleptic characteristics of the edible product while still being able to inhibit the growth of pathogens.

It has been surprisingly discovered that wildtype lactic acid bacteria may produce bacteriocin, metabolites and organic acids in the desired microbial-inhibiting amounts at a concentration in the supernatant from fermentation useful as preservatives. Accordingly, the supernatant of the bacteriocin-producing lactic acid bacteria are combined with an edible food substance with essentially no live Lactic acid bacteria, but which are able to inhibit growth of pathogens and food spoilage organisms while preserving the organoleptic properties of the food substance. The organoleptic properties of the food substance are not altered significantly by the presence of the supernatant from the lactic acid bacteria.

Bacteriocins have had limited use as food preservatives in general as they often have a narrow antimicrobial spectrum, e.g. nisin. Some bacteriocins are identified with a broad antimicrobial spectrum against both yeast, fungi, Gram-positive and Gram-negative bacteria. These broad spectrum bacteriocins has a potential as food preservatives, however typically when the amount of active bacteriocin retrieved by fermentation, the supernatant does also include multiple substances which are effecting the organoleptic properties of food products. Thus, the bacteriocins are therefore traditionally purified from the fermentation broth (supernatant) before use as food preservatives. Such purification may be undesirable, because it adds a significant cost to the bacteriocin as well as the additional antimicrobial activity from acids and other metabolites are easily lost during purification making the bacteriocin uncompetitive with the other food preservatives.

The supernatant of the present invention was observed to be suitable for directly use as a food preservative. Thus, the wild type strain is able to ferment a yield of bacteriocins during fermentation and preferably excrete sufficient amount to provide a supernatant comprising a broad spectrum activity against both yeast, fungi, Gram-positive and Gram-negative bacteria.

Also included in the supernatant is organic acids and other fermentation byproducts contributing to antimicrobial activity but with no significant effect on the organoleptic properties of the food product. The invention involves a composition comprising the ferment from a lactic acid bacteria without viable cell material. The use of such composition to preserve edible products without change of the organoleptic properties of the edible product.

### SUMMARY OF THE INVENTION

Thus, an object of the present invention relates to a supernatant from Lactic acid bacteria comprising a bacteriocin and organic acids.

In particular the invention relates to a supernatant comprising a bacteriocin and at least 1 organic acid and at least 1 fermentation by-product.

In particular, it is an object of the present invention to provide a composition that solves the above mentioned problems of the prior art without organoleptic changes of the edible products.

One aspect of the invention the bacteriocin belongs to the group of plantaricins.

Thus, one aspect of the invention relates to a composition comprising a plantaricin and at least one organic acid.

In yet another aspect of the invention at least 2 different bacteriocins are present in the composition.

The composition of the inventions comprises at least the following components: bacteriocin and organic acid.

The organic acid is selected from; lactic acid, succinic acid, acetic acid and propionic acid.

In another aspect of the invention the supernatant comprises 2 different bacteriocins produced from fermentation of one lactic acid bacteria, wherein the lactic acid bacteria are not genetically modified to produce bacteriocins.

And in still another aspect of the invention the composition is for preservation of edible products.

The present invention will now be described in more detail in the following.

### DETAILED DESCRIPTION OF THE INVENTION

### Definitions

Prior to discussing the present invention in further details, the following terms and conventions will first be defined:

The term “bacteriocin” refers to an antimicrobial peptide or protein produced by a bacteria that is active against microorganisms but does not harm the producing bacteria. For purposes of the present invention, bacterocins or bacterocin sources generally include antimicrobial agents suitable for use in food products. Especially preferred antimicrobial agents include “lantibiotics” (i.e., polypeptides containing lanthionine and beta-methyl lanthionine). Non-limiting examples of such lantibiotics are nisin, such as nisin A or nisin Z, or nisin analogs or related lanthionine-containing peptides, such as pediocin, lactosin, lactacins (e.g., lacticin A, lacticin B, lactacin F), camocin, enterocin, plantaricin, subtilin, epidermin, cinnamycin, duramycin, ancovenin, Pep 5, and the like, individually or in any combination thereof. Other bacterocins that are useful in the present invention include, for example, lactococcins (e.g., lactococcin A, lactococcin B, lactococcin M), leucocin, mesentericin, helvetican, acidophilucin, caseicin, and the like, individually or in any combination.

The term “plantaricin” refers to bacteriocins from Lactiplantibacillus plantarum, the major types of plantaricins includes Plantaricin A, Plantaricin E, Plantaricin F, Plantaricin J, Plantaricin K, Plantaricin C, Plantaricin D, Plantaricin W, Plantaricin T and Plantaricin S. As well as other plantaricins e.g. Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6. In particular broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154.

As used herein, the term “fermentation” means lactic acid fermentation, that is, the enzymatic decomposition of carbohydrates to form considerable amounts of lactic acid and/or other organic acids. The term “lactic acid bacteria” includes bacteria of the order Lactobacillales, and species from the genera Lactobacillus, Lactococcus, Leuconostoc, Streptococcus, and Pediococcus, and the like. Also included is the order Bifidobacteriales.

The term “supernatant” refers to the fermentation broth from fermentation of a lactic acid bacteria. The supernatant can be crude comprising both fermentation products, substrate from fermentation broth as well as cell material.

The composition according to the present invention may comprise at least one member chosen from a group comprising water, fermentation byproducts, organic acids, fatty acids, growth medium, culture energy source, buffered solution and/or a functional food ingredient.

A preferred embodiment of the present invention relates to a composition for control of microbial development of an edible substance the composition comprising comprises a supernatant comprising a bacteriocin produced from fermentation of a bacteriocin producing bacteria and at least one secondary antimicrobial agent produced from fermentation of the bacteriocin producing bacteria, wherein organoleptic characteristics of the edible substance are not significantly altered.

The term “Cell Free Supernatant” refers to the supernatant where live cells has been removed. Cell Free Supernatant (CFS) comprises less than 1000 viable CFU/ml. CFS can comprise cell material from dead cells.

In an embodiment of the present invention the composition comprises a concentration of viable bacteria below 10² CFU/g of said composition, such as below 10 CFU/g of said composition, e.g. below 1 CFU/g of said composition.

A “fermentation byproduct” may include at least one member chosen from a group comprising sorbate, propionate, benzoate, lactate, acetate and/or include at least one antimicrobial lactic acid producing bacteria metabolite chosen from a group comprising phenyllactic acid, 3-hydroxy phenyllactic acid, 4-hydroxy phenylactic acid, 3- hydroxy propan aldehyde, 1,2 propandiol, 1,3 propandiol, hydrogen peroxide, ethanol, acetic acid, 2-Hydroxyisocaproic acid, carbon dioxide, carbonic acid, propanoic acid, butyric acid, cyclic dipeptides, cyclo(L-Phe- L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid.

The term “inhibition” as used herein, means the killing of a microorganism, such as an undesired bacteria, or the control of the growth of a microorganism.

As used herein the term “shelf life” means the period of time that a food product remains saleable to retail customers.

E.g. In traditional meat processing, the shelf life of fresh meat and meat by-products is about 30 to 40 days after an animal has been slaughtered. Refrigeration of meat during this period of time largely arrests and/or retards the growth of pathogenic bacteria, and to a lesser extent, spoilage bacteria. After about 30 to 40 days, however, refrigeration is no longer able to effectively control the proliferation of spoilage bacteria below acceptable levels. Spoilage bacteria present on meat products after this time period are able to assimilate proteins and sugars on meat surfaces and begin to generate undesired by-products. Spoilage bacteria may also act to discolor meat, making such meat unappealing and undesirable for human consumption.

The term “spoilage bacteria” as used herein refers to any type of bacteria that acts to spoil food. Spoilage bacteria may grow and proliferate to such a degree that a food product is made unsuitable or undesirable for human or animal consumption. Bacteria are able to proliferate on food surfaces by assimilating sugars and proteins on such surfaces. By metabolizing these components, spoilage bacteria create by-products including carbon dioxide, methane, nitrogenous compounds, butyric acid, sulfur compounds, and other undesired gases and acids often makes such food unsalable to consumers.

In addition to the control of spoilage bacteria, another significant concern in the food processing industry is controlling the growth of pathogenic bacteria. As used herein, the term “pathogenic bacteria” refers to any food poisoning organism that is capable of causing disease or illness in animals or humans. The term pathogenic bacteria will be understood to include bacteria that infect edible substances and thereby can cause disease or illness after consumption by mammals, as well as bacteria that produce toxins that cause disease or illness. The proliferation of pathogenic bacteria on food products can cause severe illness and may be deadly, as demonstrated by the number of human fatalities caused by food poisoning. The term “undesired bacteria” as used herein, refers to both spoilage and pathogenic bacteria.

The term “edible substance” or “edible product” refers to a substance or product safe to oral consumption by humans or animals, which encompass food and feed products as well as ingredients for food or feed products.

The term “food product” as used herein refers to any food that is susceptible to spoilage as a result of microbial growth and proliferation on the surface of the food. Such food products include, but are not limited to meat, vegetables, fruits and grains.

The vegetable according to the present invention may relate to a vegetable- or plant-based product. The vegetable- or plant-based product may be a vegan meat product or a vegetable meat product or a plant meat product. Vegan meat product are food products that does not comprise any animal ingredients, like whey, casein, animal protein, or eggs. Vegetable meat products or a plant meat products may be products based on vegetables or plants including some animal ingredients, preferably small amounts of e.g. whey, casein, animal protein, or eggs.

As used herein, the term “meat” refers to any fresh meat product, processed meat or meat by-product from an animal of the kingdom Animalia which is consumed by humans or animals, including without limitation meat from bovine, ovine, porcine, poultry, frog, fish and crustaceous seafood. Thus, while one of the primary uses for the present invention relates to meat processed in the slaughtering of mammals in a meat processing facility, it is to be expressly understood that the invention has application in the processing of other edible meat products including fish, poultry and seafood as well as cultured meat. Moreover, it is contemplated that the method also will have use in connection with the preservation of non-animal food products, such as fruits, vegetables and grains, subject to spoilage by microorganisms.

In a preferred embodiment the meat is cultured meat. Besides “cultured meat”, the terms artificial meat, meat substitute, meat analogue, healthy meat, slaughter-free meat, in-vitro meat, vat-grown, lab-grown meat, cell-based meat, clean meat, cultivated meat and synthetic meat have all been used by various outlets to describe such product. Typically cultured meat is grown from steam cells. Some of the first descriptions of cultured meat is in U.S. Pat. No. 6,835,390 B1 with the production of tissue-engineered meat for human consumption, wherein muscle and fat cells would be grown in an integrated fashion to create food products such as beef, poultry and fish.

It will be clear to those skilled in the art that here, as well as in all the statements of range given in the present invention, characterized by such terms as “about” or “approximately,” that the precise numerical range need not be indicated with expressions such as “about” or “approx.” or “approximately,” but instead even minor deviations up or down with regard to the number indicated are still within the scope of the present invention.

A “mammal” include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; piglets; sows; poultry; turkeys; broilers; minks; goats; cattle; horses; and non-human primates such as apes and monkeys.

According to yet another embodiment of the present teachings, the fermentation byproduct includes at least one bacteriocin that is a lantibiotic and/or a non-lantibiotic. According to another embodiment of the present teachings, the fermentation byproduct includes at least one further bacteriocin selected from a group comprising nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, BHT-Aa, BHT Ab, salivarcin A5, salivarcin B, streptin, salivaricin Al, streptin, streptococcin A-FF22, mutacin BNY266, mutacin 1140, mutacin K8, mutacin II, smbAB, bovicin HJ50, bovicin HC5, macedocin, leucocin C, sakacin 5X, enterocin CRL35/mundticin, avicin A, mundticin I, enterocin HF, bavaricin A, ubericin A, leucocin A, mesentericin Y105, sakacin G, curvacin A/sakacin A, lactocin 5, cyctolysin, enterocin A, divercin V41, divercin M35, bavaricin, coagulin, pediocin PA-1, mundticin, piscicocin CS526, piscicocin 126/Vla, sakacin, Pcarnobacteriocin BM1, enterocin P, piscicoin Vlb, penocin A, bacteriocin 31, bacteriocin RC714, hiracin JM79, bacteriocin T8, enterocin SE-K4, carnobacteriocin B2 and Plantaricins.

The invention also provides for a food mixture which contains an edible food substance combined with a supernatant from a bacteriocin-producing lactic acid bacteria. The food mixture contains a CFS of the lactic acid bacteria which is effective to provide bacteriocin and/or organic acids and/fermentation byproducts at a level which will inhibit growth of a food spoilage or pathogenic organism in the food mixture. The organoleptic characteristics of the edible substance are not significantly altered by the presence of the supernatant.

The present invention includes a method for preserving food products, such as meat, by inoculating such food products with an effective amount of supernatant. The supernatant used in the present invention create essentially no malodours or discoloration of food products, such as meat, and thus act to extend the shelf life of foods products. The present invention specifically is applicable to the preservation of meat from poultry, beef, pork, lamb, fish and seafood, cultured meat as well as to dairy products, vegetables, fruits and grains.

The method is useful in inhibiting the growth of any foodborne pathogen and/or food spoilage organism, including psychrotrophic bacteria, which may be a contaminant in the food substance, including such pathogens as Listeria monocytogenes, Staphylococcus aureus, Clostridium perfringens, Clostridium botulinum, Escherihia coli, Salmonella, Bacillus cereus, and the like, and food spoilage organisms such as Streptococcus faecalis, Leuconostoc mesenteroides, Pseudomonas putrefaciens and the like. By inhibiting the growth of food spoilage organisms, the method may be used to extend the shelf-life of a food product.

A preferred embodiment of the present invention the bacteriocin produced by the lactic acid bacteria is a plantaricin selected from the group consisting of broad spectrum bacteriocins, including bacteriocins such as Plantaricin F, Plantaricin DL3, Plantaricin ZJ008, Plantaricin MG, Plantaricin Q7, Plantaricin KL-1Y, Plantaricin 163, Plantaricin 154.

A preferred embodiment of the present invention the bacteriocin produced by the lactic acid bacteria is a plantaricin F.

A preferred embodiment of the present invention at least two different bacteriocins are produced by the lactic acid bacteria.

A preferred embodiment of the present invention the two different bacteriocins produced by the lactic acid bacteria is two different plantaricins.

A preferred embodiment of the present invention the different bacteriocins produced by the lactic acid bacteria is a plantaricin F and at least one more plantaricin.

A preferred embodiment of the present invention at least 3 different bacteriocins are produced by the lactic acid bacteria.

A preferred embodiment of the present invention at least 4 different bacteriocins are produced by the lactic acid bacteria.

In another aspect, the present teachings disclose a substantially pathogen-free and/or spoilage-microorganism-free edible composition. The substantially pathogen-free and/or spoilage-microorganism-free edible composition includes: (i) application including: (a) cell free supernatant from a bacteriocin producing lactic acid bacteria; and (b) a fermentation byproduct produced from the fermentation; and (ii) an edible product that includes the cell free supernatant.

The substantially pathogen-free and/or spoilage-microorganism-free edible composition may also include at least one member chosen from a group comprising flavor enhancer, palatant, salts, stabilizing agent, food coating stabilizer, fragrance, binder, color, and coloring agent.

Preferably, the substantially pathogen-free and/or spoilage-microorganism-free edible composition includes less than about 10000 CFU of a pathogen and/or a food-spoilage microorganism per gram of the edible product. More preferable less than about 1000 CFU of a pathogen and/or a food-spoilage microorganism per gram of the edible product. More preferable less than about 100 CFU of a pathogen and/or a food-spoilage microorganism per gram of the edible product. More preferable less than about 10 CFU of a pathogen and/or a food-spoilage microorganism per gram of the edible product. Fresh meat is known to have a pH of about 5.3 to about 7. At pH levels of below about 4 the majority of spoilage and pathogenic bacteria are either killed or their growth is severely inhibited and/or arrested. Contacting fresh meat with an effective amount of a weak organic acid, such as acetic or lactic acid, lowers the pH of the meat from about pH 3 to about pH 5 and preferably to about 4. The acidification of the surface of red meat also has other beneficial effects. Organic acids act to maintain meat in a reduced state, thereby maintaining a desirable red color of the meat. The present invention therefore includes a method for creating an acidic environment on the surface of a meat product, processed meat, cultured meat or useful in ground or minced meat or cultured meat products incorporating vegetative matter, such as oat flour.

This invention is based upon the discovery that some species of lactic acid bacteria will produce bacteriocin in the supernatant in an amount effective to inhibit growth of foodborne pathogens and food spoilage organisms even though the lactic acid bacteria are no longer present and there will be no fermentation of the edible substance.

The invention provides a method of inhibiting the growth of food spoilage and/or foodborne pathogenic organisms in edible food substances by combining the food substance with CFS comprising bacteriocins without producing detectable flavor, aroma, textural or other organoleptic changes in the edible substance.

According to the invention, preferred bacteriocin-producing lactic acid bacteria are from the phylogenic order Lactobacillales, more preferably the bacteriocin-producing lactic acid bacteria is selected from the families; Lactobacillus, Paralactobacillus, Acetilactobacillus, Agrilactobacillus, Amylolactobacillus, Apilactobacillus, Bombilactobacillus, Companilactobacillus, Dellaglioa, Fructilactobacillus, Furfurilactobacillus, Holzapfelia, Lacticaseibacillus, Lactiplantibacillus, Lapidilactobacillus, Latilactobacillus, Lentilactobacillus, Levilactobacillus, Ligilactobacillus, Limosilactobacillus, Liguorilactobacillus, Loigolactobacilus, Paucilactobacillus, Schleiferilactobacillus, and Secundilactobacillus.species.

In a preferred embodiment the bacteriocin producing lactic acid bacteria is Lactiplantibacillus.

According to the invention, it is preferred that the CFS does not significantly alter the pH or the organoleptic characteristics such as flavor, aroma, color, or texture of the edible substance.

To optimize bacteriocin production by the lactic acid bacteria and the growth inhibitory activity of the bacteriocin in the food mixture, it is preferred that the pH of the food mixture is maintained at about pH 4-8, more preferably a pH of about 4.5-6. The pH may be maintained at the desired level, as for example, by inherent buffers in the food mixture.

Bacteriocins are generally known as being effective in inhibiting pathogenic and spoilage microorganisms in foods, such as described by Twomey, D. et al., Lantabiotics Produced by Lactic Acid Bacteria: Structure, Function and Applications, Antonie van Leeuwenhoek, 82:15-185, 2002, and Cleveland, J., et al., “Bacteriocins: Safe, Natural Antimicrobials for Food Preservation,” Int'l J. Food Micro., 71 (2001) 1-20. Bacteriocins are generally understood to act on sensitive cells by forming pores in the cytoplasmic membrane. This leads to the dissipation of the proton motive force and release of small intracellular molecules like glutamate and ATP, such as described by Twomey et al. and Cleveland et al., referenced above. This renders the cells permeable but still capable of participating in biochemical processes in its environment. The treatment of cells with surface-active agents to help generate such “leaky” cells, has been described in PCT Int'l Publication No. WO 01/47366 Al. This activity is typically obtained by the purified bacteriocins or by co-growth of the bacteriocin producing bacteria with the pathogenic and spoilage microorganisms.

In the present invention, at least one secondary antimicrobial agent may be included in the CFS in combination with the bacterocin.

The composition according to the present invention may comprise bacteriocin, an organic acid, and one or more metabolites (such as at least one secondary antimicrobial agent) produced from fermentation of the bacteriocin producing bacteria.

The organic acid may be selected from lactic acid, succinic acid, acetic acid, propionic acid, or 2-Hydroxyisocaproic acid.

In an embodiment of the present invention the composition consists essentially of bacteriocin, organic acid, and metabolites (such as at least one secondary antimicrobial agent) produced from fermentation of the bacteriocin producing bacteria.

The at least one secondary antimicrobial agent produced from fermentation of the bacteriocin producing bacteria may be selected from one or more of metal chelating agents, organic acids, fatty acids, short chain free fatty acids.

In an embodiment of the present invention the composition comprises a bacteriocin produced from fermentation of the bacteriocin producing bacteria, an organic acid produced from fermentation of the bacteriocin producing bacteria, and at least one metabolite (such as at least one secondary antimicrobial agent) produced from fermentation of the bacteriocin producing bacteria.

In a further embodiment of the present invention the composition does not comprise added organic acids and/or added fatty acids and/or added salts.

The at least one secondary antimicrobial agent may preferably be produced from fermentation of the bacteriocin producing bacteria.

Examples of such secondary antimicrobial agents may include one or more of metal chelating agents (e.g., citric acid, and the like), organic acids, 2-Hydroxyisocaproic acid, short chain free fatty acids, proton ionophores (e.g., sorbic acid, benzoic acid, and the like), lacto-antimicrobials (e.g., lactoferrin, lactolipids, and the like), monoglycerides (e.g., monolinolenin, monolaurin, and the like), hops acids, and the like. When used, these secondary antimicrobial agents are generally present at levels of about 0.01 to about 0.5 percent. For organic acids the concentration in the CFS is higher at a about 0.5 to 7%.

According to one embodiment of the present invention the fermentation byproduct is a short chain fatty acid (SCFA) selected from formic acid, acetic acid, propanoic acid, butanoic acid, pentanoic acid, hexanoic acid, heptanoic acid, 2-methyl-propanoic acid, 2-Hydroxyisocaproic acid, 3-methyl-butanoic acid, 4-methyl-pentanoic acid.

Preferred combinations include bacteriocin and organic acids.

Preferred combinations include at least one bacteriocin and at least one organic acids and at least one SCFA.

A fermentation byproduct may include at least one member chosen from a group comprising bacteriocins, plantaricins, hydrogen peroxide, lipoteichonic acids, teichonic acids, salts, glycoprotein, and acid mucin.

According to one embodiment of the present teachings, the fermentation byproduct includes at least one antimicrobial lactic acid producing bacterial metabolite chosen from a group comprising phenyllactic acid, 3-hydroxyphenyllactic acid, 4-hydroxyphenylactic acid, 3 -hydroxy propanaldehyde, 1,2 propandiol, 1,3 propandiol, hydrogen peroxide, ethanol, acetic acid, carbon dioxide, carbonic acid, propanoic acid, butyric acid, 2-Hydroxyisocaproic acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3- (R)-hydroxy dodecanoic acid, and 3-(R)-hyroxytetradecanoic acid.

According to another embodiment of the present teachings, the fermentation byproduct include at least one bacteriocin that is a lantibiotic (Class II) or a non-lantibiotic (Class II). According to yet another embodiment of the present teachings, the fermentation byproduct include at least one bacteriocin selected from a group comprising Plantaricin A, Plantaricin E, Plantaricin F, Plantaricin J, Plantaricin K, Plantaricin C, Plantaricin D, Plantaricin W, Plantaricin T, Plantaricin S, Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6. In particular broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154, nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, salivarcin A5, salivarcin B, streptin, salivaricin Al, streptin, streptococcin A-FF22, BHT-Aa, BHT Ab, mutacin BNY266, mutacin 1140, mutacin K8, mutacin II, smbAB, bovicin H350, bovicin HC5, macedocin, plantaricin W, lactocin 5, cyctolysin, enterocin A, divercin V41, divercin M35, bavaricin, coagulin, pediocin PA-1, mundticin, piscicocin CS526, piscicocin 126/Vla, sakacin P, leucocin C, sakacin 5X, enterocin CRL35/mundticin, avicin A, mundticin I, enterocin HF, bavaricin A, ubericin A, leucocin A, mesentericin Y105, sakacin G, plantaricin 423, plantaricin C 19, curvacin A/sakacin A, carnobacteriocin BM1, enterocin P, piscicoin Vlb, penocin A, bacteriocin 31, bacteriocin RC714, hiracin JM79, bacteriocin T8, enterocin, or carnobacteriocin.

In one preferred embodiment the CFS comprises at least 2 plantericins selected from the group; Plantaricin A, Plantaricin E, Plantaricin F, Plantaricin J, Plantaricin K, Plantaricin C, Plantaricin D, Plantaricin W, Plantaricin T, Plantaricin S, Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6.

In particular at least one plantaricin selected from the group of broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154.

In an embodiment of the present invention the plantaricin is one or more of plantaricin E, plantaricin F, plantaricin A, and/or plantaricin J.

Preferably, the plantaricin relates to a combination of 2 or more of plantaricin E, plantaricin F, plantaricin A, and/or plantaricin J; such as 3 or more, e.g. all 4 of plantaricin E, plantaricin F, plantaricin A, and/or plantaricin J.

In one embodiment the preferred bacteriocin used in the present invention is from fermentation of one of the following bacteriocin-producing bacteria; Weissella viridescens LB10G (DSM 32906), Lacticaseibacillus paracasei LB113R (DSM 32907), Lactiplantibacillus plantarum LB244R (DSM 32996), Lacticaseibacillus paracasei LB116R (DSM 32908), Levilactibacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB356R (DSM 33094), Lactiplantibacillus plantarum LB312R (DSM 33098); and/or any combinations hereof.

The preferred bacteriocin used in the present invention is Plantaricin F. Plantaricin F is produced by e.g. Lactiplantibacillus plantarum LB244R deposited with DSMZ (DSM32996) and e.g. Lactiplantibacillus plantarum LB356R deposited with DSMZ (DSM33094)

The easiest method for providing the CFS comprising bacteriocin, such as plantaricin, is to dry the CFS containing the bacteriocin after fermentation to produce a powder or a concentrated slurry.

The solid materials can be removed after fermentation by filtration or centrifugation from the growth medium. Low molecular weight compounds can be removed by membrane filtration, particularly reverse osmosis. Food grade drying aids such as non-fat dry milk (NFDM) can be used to dry the solution containing the bacteriocin. The bacteriocin is a proteinaceous material and can also be separated or isolated from the growth medium by precipitation or by other well known techniques such as reverse osmosis and it can then be dried in a pure form.

In one embodiment of the invention the CFS is concentrated.

In a further embodiment of the present invention, the CFS does not comprise a separated, isolated, or purified bacteriocin. In one embodiment of the invention the CFS is concentrated without any separation, isolation or purification.

In a further embodiment of the present invention the composition or the CFS comprises minerals and/or salts from fermentation broth.Concentrating may include separating an amount of the fluid portion from the fermented growth culture using at least one technique chosen from a group comprising filtering, sedimenting, centrifuging, vacuuming, decanting, drying, freeze drying, spray drying, and evaporating. The method for producing the cell free supernatant may further include drying the fermented cell free supernatant.

In one preferred embodiment the CFS is concentrated by removing water.

In one preferred embodiment the CFS is concentrated 2 times by removing water.

In one preferred embodiment the CFS is concentrated 3 times by removing water.

In one preferred embodiment the CFS is concentrated more than 2 times by removing water.

In one preferred embodiment the CFS is concentrated by removing water to obtain a dried powder.

The bacteriocin is preferably used in the edible substance in an amount between 1 and 1,000,000 Arbitrary Units (AU) of bacteriocin, such as PA-1, per gram of the food. Once AU of bacteriocin was defined as 5 microliters of the highest dilution of culture supernatant yielding a definite zone of growth inhibition with a lawn of an indicator strain of a gram-positive bacteria on an agar plate.

The organic acid is preferable used in the edible substance in a concentration by weight from about 1 to 7%. E.g. by weight from, from 1 to 2 percent lactic acid and from 1.5 to 3.0 percent organic acid.

The organic acid is preferable selected from lactic acid, acetic acid, malic acid, tartaric acid, propionic acid, 2-Hydroxyisocaproic acid.

Further components which optionally can be added to the CFS is salts or salt-like substance may include at least one member chosen from a group comprising sodium chloride, potassium chloride, sea salt, and calcium chloride. According to another embodiment of the present teachings, a binder and/or a syneresis controlling substance is added. A binder, stabilizer or a syneresis controlling substance may be at least one member chosen from a group comprising pea powder, gum arabic, guar gum, hydrocolloids, carboxymethylcellulose, locust bean gum, cassia gum, carageenans, iota-carageenan, kappa-carageenan, milk, milk products, milk proteins, casein, pork plasma, textured vegetable proteins, glutens, corn gluten, wheat gluten, starches, corn starch, rice starch, potato starch, tapioca starch, sorghum starch, oat starch, soy, soy protein, soy protein concentrate, soy protein isolate, egg, egg derivatives, transglutaminase, gelatins, and polysaccharides. In particular, such substances may be useful in high-moisture foods for mitigating the effects of syneresis that occur with e.g. meat-based food products. A buffering agent (e.g., calcium carbonate, sodium bicarbonate) may be used to stabilize a food such as meat, vegetables, fruit, pet food, pet treats or any mixture thereof.

The CFS composition for preservation may advantageously further comprise other probiotics, prebiotics, or other active substances and/or may preferably also contain one or more of the following substances selected from antioxidants, vitamins, coenzymes, fatty acids, amino acids and cofactors.

A preferred embodiment of the present invention relates to a composition for control of microbial control development of an edible substance comprising a supernatant comprising a bacteriocin produced from fermentation of a bacteriocin producing bacteria wherein organoleptic characteristics of the edible substance are not significantly altered.

Preferably the concentration of viable cells in the supernatant may be below 102 CFU/g of said composition, such as below 10 CFU/g of said composition, e.g. below 1 CFU/g of said composition.

In an embodiment of the present invention the bacteriocin in the supernatant is concentrated to an amount at least 2 times greater than said original amount after fermentation.

In an embodiment of the present invention the bacteriocin producing bacteria is selected from a group comprising Lactobacillus plantarum, Lactobacillus acidophilus, Lactobacillus reuteri, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), Oxalobacter formagenes, Bifidobacterium bifidus, and Leuconostoc mesenteroides.

A preferred embodiment of the present invention relates to an edible substance comprising a food product or a feed product, and a composition comprising a supernatant produced from fermentation of a bacteriocin producing bacteria.

The present invention may relate to an edible substance comprising a food product or a feed product, and a composition consisting essentially of a supernatant produced from fermentation of a bacteriocin producing bacteria.

The supernatant comprises substantially no bacteriocin producing bacteria.

In an embodiment of the present invention wherein the concentration of pathogenic microorganism, in particular pathogenic bacteria, may be below 103 CFU/gram edible substance, such as below 10² CFU/gram edible substance, e.g. below 10 CFU/gram edible substance, preferably, the concentration of pathogenic microorganism, in particular pathogenic bacteria, may not be detectable.

The supernatant may comprise a pH value in the range of 2.5-6.5, such as in the range of pH 3.0-6.0, e.g. in the range of 3.5-5.5, such as in the range of pH 4-5.

In an embodiment of the present invention a salt may be added to the fermentation broth during fermentation to facilitate fermentation of the bacteriocin producing bacteria. The salt added may preferably be sodium chloride (NaCl).

Salt may be added to the fermentation broth may be in a concentration of less than 2% (w/w), such as in a concentration less than 1.5% (w/w), e.g. in a concentration less than 1% (w/w).

In an embodiment of the present invention the salt concentration of the supernatant may be less than 2% (w/w), such as less than 1.5% (w/w), e.g. less than 1% (w/w), such as less than 0.75% (w/w), e.g. less than 0.5% (w/w).

In a further embodiment of the present invention the concentration of pathogenic microorganism in the edible substance, in particular pathogenic bacteria, may be reduced by at least 10% relative to the originally concentration of pathogenic microorganisms in the edible substance, such as by at least 20%, e.g. by at least 30%, such as by at least 40%, e.g. by at least 50%, such as by at least 60%, e.g. by at least 70%, such as by at least 80%, e.g. by at least 90%, such as by at least 95%, e.g. by at least 98%.

The pathogenic microorganism may be E. Coli, staphylococcus (like S. enterica, S. aureus), Campylobacter, Clostridium (like Clostridium botulinum), Listeria (like Listeria monocytogenes), Salmonella, Shigella, or a combination hereof. Preferably, the pathogenic microorganism may be E. Coli, staphylococcus (like S. enterica, S. aureus), or a combination hereof.]

An embodiment of the present invention relates to an edible substance preserved by a composition comprising a supernatant produced from fermentation of a bacteriocin producing bacteria wherein organoleptic characteristics of the edible substance are not significantly altered.

In an embodiment of the present invention the edible substance comprises a substantially pathogen-free and/or spoilage-microorganism-free food composition according of the present invention. preferably, the edible substance comprises less than about 10 cfu of a pathogen and/or a food spoilage microorganism per gram of said edible substance.

Preferably, the composition according to the present invention may be, or be used as, a food preservative.

An embodiment of the present invention relates to a method for producing a food preservative, said method comprising the steps of:
(i) mixing a bacteriocin producing bacteria in a growth culture comprising a growth medium and an energy source;
(ii) fermenting said bacteriocin producing bacteria in the presence of said growth culture to produce a fermented growth culture comprising a bacteriocin producing bacteria and a bacteriocin;
(iii) removing the bacteriocin producing bacteria to obtain a substantially cell free supernatant; and
(iv) optionally, concentrating the bacteriocin in the supernatant.

In an embodiment of the present invention the bacteriocin may be secreted by the bacteria grown into the growth culture.

Preferably, the supernatant of the present invention may be provided without disruption, lysis, or degradation of the bacteriocin producing bacteria. By avoiding disruption of the bacteriocin producing bacteria, the concentration of bacteriocin in the supernatant may be increased and/or have stronger activity, since there are no impurities in the supernatant.

In an embodiment of the present invention the bacteriocin may be an extracellular bacteriocin.

The term “extracellular bacteriocin” relates to the bacteriocin excreted by the bacteriocin producing bacteria and available in the fermentation broth.

The supernatant according to the present invention may be concentrated by removing water without inactivation of the fermentation byproduct.

The concentrating (or the removal of water) may include separating an amount of water from said fermented growth culture using at least one technique chosen from a group comprising sedimenting, centrifuging, vacuuming, decanting, drying, freeze drying, spray drying, and evaporating.

The fermentation may preferably be carried out at a temperature between about 25 degrees centigrade and about 40 degrees centigrade.

A preferred embodiment of the present invention relates to a process for producing an edible substance, said process comprising:
(1) obtaining a food preservative and a food product, and said food preservative includes:
   (a) a supernatant from a bacteriocin producing bacteria,
   (b) a bacteriocin; and
   (c) optionally at least one further antimicrobial agent;
(2) applying said food preservative to a surface of said food product to produce an edible substance that is substantially free of pathogens and/or spoilage microorganisms.

In a preferred embodiment of the present invention the food preservative may comprise at least one metabolite produced from fermentation of the bacteriocin producing bacteria. The at least one metabolite produced from fermentation of the bacteriocin producing bacteria may comprise at least one least one secondary antimicrobial agent produced from fermentation of the bacteriocin producing bacteria. Preferably, the at least one secondary antimicrobial agent produced from fermentation of the bacteriocin producing bacteria may be selected from one or more of metal chelating agents, organic acids, fatty acids, short chain free fatty acids.

The edible substance may be a shelf-stable edible substance. Shelf-stable edible substance may be an edible substance that can be safely stored at room temperature, optionally in a sealed container. This may also include edible substances that would normally be stored refrigerated but which have been processed so that they can be safely stored at room or ambient temperature for a usefully long shelf life.

A preferred embodiment of the present invention relates to an edible substance comprising a food product or a feed product, and a composition comprising a supernatant produced from fermentation of a bacteriocin producing bacteria and an organic acid produced from fermentation of the bacteriocin producing bacteria.

A further preferred embodiment of the present invention relates to an edible substance preserved by a composition comprising a supernatant produced from fermentation of a bacteriocin producing bacteria and an organic acid produced from fermentation of the bacteriocin producing bacteria, wherein organoleptic characteristics of the edible substance are not significantly altered.

In an embodiment of the present invention wherein the term “substantially free of” relates to a content of at most 5% (w/w) of the edible substance, such as at most 4% (w/w), e.g. at most 3% (w/w), such as at most 2%(w/w), e.g. at most 1% (w/w), such as at most 0.1%(w/w), e.g. at most 0.01% (w/w).

The shelf-stable edible substance includes less than about 10 cfu of said pathogens and/or said spoilage microorganisms per gram of said shelf-stable edible substance.

In an embodiment of the present invention the food preservative may be applied to said surface of said food product at a concentration that may be between about 0.01 percent to about 10 percent by weight of said food preservative relative to said food product, such as between 0.05-9% (w/w), e.g. between 0.1-8% (w/w), such as between 0.5-7% (w/w), e.g. between 1-6% (w/w), such as between 2-5% (w/w), e.g. between 3-4% (w/w).

In a further embodiment of the present invention the food preservative may be applied to the surface of the food product using at least one technique chosen from a group comprising coating, spraying, soaking, misting, aerosolizing, affixing, and atomizing.

The food product may preferably include at least one member chosen from a group comprising meat, meat products, processed meat, raw meat, fermented meat, kibbled food, kibble, refrigerated food, refrigerated treat, frozen food, frozen treat, biscuit, raw food, treat, soft-moist food, soft-moist treat, pellet, fine, broken piece, supplement, a sauce, a juice, a meal replacement drink, a probiotic drink, prepared food, a ready-to-eat meal, a functional food, a functional beverage, a whole fruit, a whole vegetable, prepared salad ingredient, ground fruit, grounded vegetable, prepared meal, slaughtered carcass, prepared food, meat piece, meat chunk, fabricated meat chunk, fabricated protein chunk, livestock feed, steam-flaked feed, and aquaculture feed.

The edible substance may have a pH below 6, such as a pH below 5, e.g. a pH below 4.5, such as a pH below 4.

In an embodiment of the present invention the method of providing the edible substance may involve packaging of the edible substance providing a packed edible substance.

A further embodiment of the present invention relates to a method of inhibiting the growth of a food spoilage or pathogenic organism in an edible substance, comprising: combining a food product, and a food preservative, wherein the food preservative comprises a bacteriocin and a metabolite according to the present invention, a second at least one secondary antimicrobial agent according to the present invention, such as a one or more of metal chelating agents, organic acids, fatty acids, short chain free fatty acids, in particular an organic acid, and wherein the food preservative is incapable of significantly fermenting the food product, and the food preservative is effective in providing the bacteriocin to the edible substance to inhibit growth of the food spoilage or pathogenic organism in the edible substance.

Preferably, the organoleptic characteristics of the edible substance may not be significantly altered by the presence of the food preservative.

The edible substance may be kept at a temperature of about 1-7 degrees centigrade.

The edible substance may comprise a pH of about pH 4.5 to less than about pH 7.

All patent and non-patent references cited in the present application, are hereby incorporated by reference in their entirety.

The invention will now be described in further details in the following non-limiting examples.

### EXAMPLES

### Example 1

Growth inhibition was measured by contrast phase microscopy and image analysis using the oCelloscope (BioSense Solution, Denmark). The inhibitory effect of Cell Free Supernatant (CFS) on selected pathogens was measured for and various lactic acid bacteria (LAB) strains was tested according to Fredborg et al. with modifications (Fredborg, M., Andersen, K. R., Jørgensen, E., Droce, A., Olesen, T., Jensen, B. B., et al. (2013) ‘Real-Time Optical Antimicrobial Susceptibility Testing’, Journal of Clinical Microbiology, 51(7), pp. 2047-2053. doi: 10.1128/JCM.00440-13.). Overnight culture of the pathogenic test organism was diluted to a concentration of approx. 10⁴ CFU/ml. The overnight culture of LAB (10⁹ CFU/ml) was filtered through a 0.2 μm filter to remove all cells. The CFS was diluted into 75%, 50%, 25% and 10%. A 100 μL aliquot of diluted pathogen cell suspension was mixed with 100 μL undiluted or diluted CFS in 96 well plates. The plate was sealed with oxygen penetrating film cover (Sigma-Aldrich) and incubated in the oCelloScope instrument (BioSense Solution, Denmark) at 37° C. for 18 hours. The pathogen growth is measured every 20 min as segmentation and extraction of surface area (SESA).

The following test pathogens/spoilage microorganisms were used:

Eschericia coli CCUG 11775

Salmonella enterica CCUG 43791

Staphylococcus aureus MRSA USA300

CFS from the following lactic acid bacteria with antimicrobial activity were tested: Lactiplantibacillus plantarum LB244R (DSM 32996), Lactiplantibacillus plantarum LB316R (DSM 33091), Lactiplantibacillus plantarum LB356R (DSM 33094), Lactiplantibacillus plantarum LB312R (DSM 33098), Lacticaseibacillus paracasei LB116R (DSM 32908), Lacticaseibacillus paracasei LB113R (DSM 32907), Lacticaseibacillus paracasei LB28R (DSM 32994), Enterococcus faecium LB276R (DSM 32997), Leuconostoc mesenteriodes LB349R (DSM 33093), Weissella viridescens LB10G (DSM 32906) and Levilactobacillus brevis LB152G (DSM 32995)

The Minimum Inhibitory Concentration (MIC) was determined as the most diluted concentration of CFS still being able to growth inhibited the respective pathogen (table 1).

TABLE 1
MIC of the CFS, lowest dilution of CFS able
to growth inhibit the pathogen (% dilution).

| | Pathogen | | |
|---|---|---|---|
| LAB | E. coli | S. enterica | S. aureus |
| LB244R | 10% | 10% | 10% |
| LB316R | 50% | 75% | 75% |
| LB356R | 10% | 10% | 10% |
| LB312R | 25% | 10% | 25% |
| LB113R | 50% | 50% | 25% |
| LB116R | 25% | 25% | 25% |
| LB276R | 10% | 25% | 25% |
| LB349R | 10% | 25% | 25% |
| LB10G | 10% | 10% | 10% |
| LB152G | 75% | 50% | 50% |

### Example 2

The bacteriocins in the two most active strains were identified by sequencing. Whole genome sequenced by Baseclear (Leiden, Netherlands) and annotated by servers such as Rapid Annotation Subsystem Technology (RAST) server (http://rast.nmpdr.org) and the annotation program Bacteriocin Genome mining tool, BAGEL4 (http://bagel4.molgenrug.nl/index.php) to reveal potential bacteriocin encoding genes and for virulence or disease encoding genes. Subsequently, the genome sequence of LB244R and LB356R was annotated by Baseclear.

Several genes involved in bacteriocin production were identified in the LB244R and LB356R genome sequences (Table 2).

| LAB | Identified bacteriocins |
|---|---|
| Lactiplantibacillus plantarum LB244R | Plantaricin E |
| | Plantaricin F |
| | Plantaricin A |
| | Plantaricin J |
| | Enterocin |
| Lactiplantibacillus plantarum LB356R | Plantaricin E |
| | Plantaricin F |
| | Plantaricin A |
| | Plantaricin N |
| | Plantaricin J |
| | Plantaricin K |

### Example 3

### Metabolomics for Fermentation By-Products

Metabolomics were done on 3 different media fermentations. Bacteriocin producing Lactic acid bacteria LB244R and LB356R were grown in different growth media (1: malted barley, 2: wheat, 3: barley, 4: barley short extraction 25 min) based on carbohydrate water extraction 1 hour from either barley or whey at 75° C., sterilized by autoclavation and filtered. Fermented at different conditions (1: 30° C. at 48 hours, 2: 30° C. at 48 hours followed by preservation, 3: 30° C. at 48 hours pH adjusted to 5.5).

Smell and taste of the different fermentations were evaluated to be neutral. The supernatant analysed by the semi-polar metabolites method. Sample analysis was carried out by MS-Omics (Vedbæk, Denmark) as follows.

The samples were diluted 10 times in 10 mM ammonium formate with 0.1% formic acid.

### LC-MS Method

The analysis was carried out using a UPLC system (Vanquish, Thermo Fisher Scientific) coupled with a high-resolution quadrupole-orbitrap mass spectrometer (Q Exactive™ HF Hybrid Quadrupole-Orbitrap, Thermo Fisher Scientific). An electrospray ionization interface was used as ionization source. Analysis was performed in negative and positive ionization mode. A QC sample was analysed in MS/MS mode for identification of compounds. The UPLC was performed using a slightly modified version of the protocol described by Catalin et al. (UPLC/MS Monitoring of Water-Soluble Vitamin Bs in Cell Culture Media in Minutes, Water Application note 2011, 720004042en).

### Data Processing

Data was processed using Compound Discoverer 3.1 (ThermoFisher Scientific) and TraceFinder 4.1 (ThermoFisher Scientific).

### Compound Extraction

One compound often gives rise to a signal in more than one mass trace (due to e.g. naturally occurring C13 isotopes, adducts, and/or fragments) a compound will therefore almost always be represented by more than one feature with the same retention time but different masses. The compound extraction performed by Compound Discoverer consists of the following four steps:
1) First, features are extracted from the raw data.
2) The feature detection is followed by grouping of features belonging to the same compound.
3) This additional information (e.g. isotope pattern) is then used together with the accurate mass to determine the molecular formula.
4) The total information collected for each compound are then used in the following identification step.

The analysis was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Catalin et al. (UPLC/MS Monitoring of Water-Soluble Vitamin Bs in Cell Culture Media in Minutes, Water Application note 2011, 720004042en). Peak areas were extracted using Compound Discoverer 3.1 (Thermo Scientific).

Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm).

Level 2b: identification by accurate mass (with an accepted deviation of 3 ppm), and MS/MS spectra, Level 3: identification by accurate mass alone (with an accepted deviation of 3 ppm). A total of 1964 compounds were detected in the samples. Hereof were 115 annotated on level 3, 174 on level 2b, 85 on level 2a, and 93 on level 1.

Lactic acid, acetic acid, succinic acid, salicylic acid, indole-3-lactic acid, indole-3-acetic acid, 2-hydroxybuturic acid, 2-Hydroxyisocaproic acid and N-acetylaspartic acid were all annotated at level 1 in significant amounts.

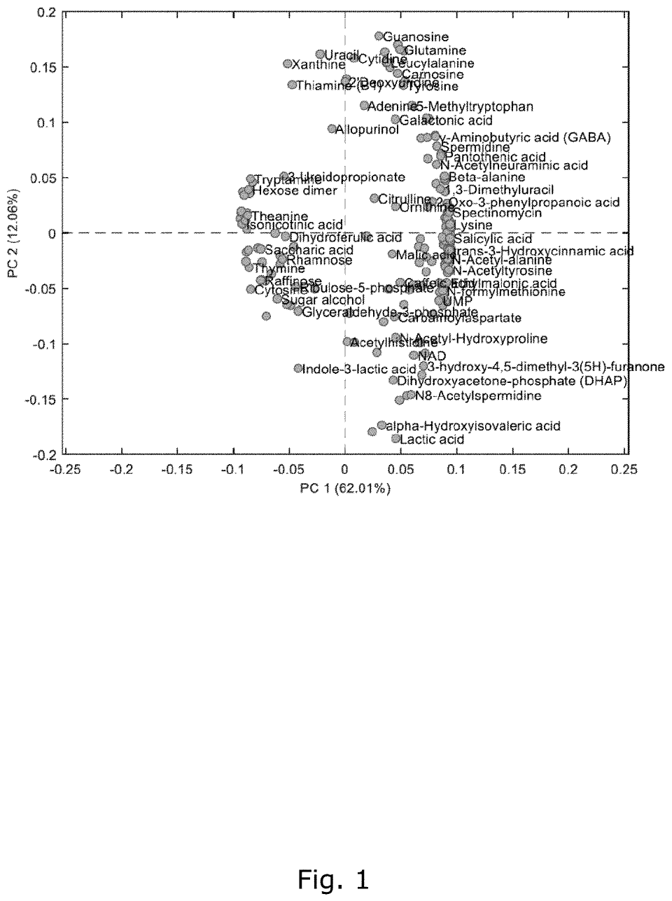

SCFA were determined and present at level 1 was acetate, butyrate og propionate. Loading plot from PCA model calculated for metabolites annotated at level 1 and 2a (FIG. 1).

### Example 4

### Food Preservation Testing

Minced raw fresh meat (tartare) 90 g and 10 g of CFS were mixed, 20 small meatballs of approximately 5 g were incubated at either 5° C. or 20° C. For the meatballs at 5° C. samples were taken every second or third day for 4 weeks and for the meatballs at 20° C. samples were taken every day for 5 days. As control for meat without CFS, 10 g sterile water was used. Each sample were tested for total viable count by total plate counting on Tryptone Soya Agar (TSA) and incubation at 30° C. as well as an organoleptic evaluation was performed scoring on a scale from 1 to 3 for each parameter smell, color and appearance. With 1 being the score of fresh meat and 3 being a score for spoiled meat.

CFS from the following lactic acid bacteria with antimicrobial activity were tested: Lactiplantibacillus plantarum LB244R (DSM 32996), Lactiplantibacillus plantarum LB316R (DSM 33091), Lactiplantibacillus plantarum LB356R (DSM 33094), Lactiplantibacillus plantarum LB312R (DSM 33098), Lacticaseibacillus paracasei LB116R (DSM 32908), Lacticaseibacillus paracasei LB113R (DSM 32907), Lacticaseibacillus paracasei LB28R (DSM 32994), Enterococcus faecium LB276R (DSM 32997), Leuconostoc mesenteriodes LB349R (DSM 33093), Weissella viridescens LB10G (DSM 32906) and Levilactobacillus brevis LB152G (DSM 32995)

At 5° C. the CFS from bacteriocin-producing lactic acid bacteria were able to preserve the fresh meat for 5-11 days longer than the meat without CFS. The longest preservation without any organoleptic changes were observed for Lactiplantibacillus plantarum LB244R and Lactiplantibacillus plantarum LB316R.

1. Composition for control of microbial development of an edible substance the composition comprises a supernatant comprising a bacteriocin produced from fermentation of a bacteriocin producing bacteria and an organic acid produced from fermentation of a bacteriocin producing bacteria, wherein organoleptic characteristics of the edible substance are not significantly altered.

2. Composition of claim 1, wherein said bacteriocin in the supernatant is concentrated to an amount at least 2 times greater than said original amount after fermentation.

3. Composition according to claim 1, wherein the bacteriocin producing bacteria is a lactic acid bacteria.

4. Composition according to claim 1, wherein the composition comprises minerals and/or salts from fermentation broth.

5. Composition according to claim 1, wherein the organic acid may be selected from lactic acid, succinic acid, acetic acid, propionic acid, or 2-Hydroxyisocaproic acid.

6. Composition according to claim 1, wherein the composition comprises one or more metabolites from fermentation of the bacteriocin producing bacteria.

7. Composition according to claim 1, wherein the composition comprises one or more fatty acids produced from fermentation of the bacteriocin producing bacteria.

8. Composition according to claim 1, wherein the bacteriocin is a plantaricin.

9. Composition according to claim 8 wherein, the plantaricin is one or more of plantaricin E, plantaricin F, plantaricin A, and/or plantaricin J.

10. An edible substance comprising a food product or a feed product, and a composition comprising a supernatant produced from fermentation of a bacteriocin producing bacteria and an organic acid produced from fermentation of the bacteriocin producing bacteria.

11. An edible substance preserved by a composition comprising a supernatant produced from fermentation of a bacteriocin producing bacteria and an organic acid produced from fermentation of the bacteriocin producing bacteria, wherein organoleptic characteristics of the edible substance are not significantly altered.

12. The edible substance according to claim 10, wherein the edible substance includes less than about 10 cfu of a pathogen and/or a food spoilage microorganism per gram of said edible substance.

13. A method for producing a food preservative, said method comprising the steps of:
(i) mixing a bacteriocin producing bacteria in a growth culture comprising a growth medium and an energy source;
(ii) fermenting said bacteriocin producing bacteria in the presence of said growth culture to produce a fermented growth culture comprising a bacteriocin producing bacteria and a bacteriocin;
(iii) removing the bacteriocin producing bacteria to obtain a substantially cell free supernatant; and
(iv) optionally, concentrating the bacteriocin in the supernatant.

14. A process for producing an edible substance, said process comprising:
(1) obtaining a food preservative and a food product, and said food preservative includes:
(a) a supernatant from a bacteriocin producing bacteria,
(b) a bacteriocin;
(c) optionally at least one further antimicrobial agent; and
(d) an organic acid produced from fermentation of the bacteriocin producing bacteria;
(2) applying said food preservative to a surface of said food product to produce an edible substance that is substantially free of pathogens and/or spoilage microorganisms.

15. Method of inhibiting the growth of a food spoilage or pathogenic organism in an edible substance, comprising: combining a food product, and a food preservative, wherein the food preservative comprises a bacteriocin and an organic acid, and wherein the food preservative is incapable of significantly fermenting the food product, and the food preservative is effective in providing the bacteriocin to the edible substance to inhibit growth of the food spoilage or pathogenic organism in the edible substance.
